(19) United States
(12) Patent Application Publication
(10) Pub. No.: US 2013/0041339 A1
(43) Pub. Date: Feb. 14, 2013

(54) Panty Pads

(71) Applicant: Tamara Taylor, Memphis, TN (US)

(72) Inventor: Tamara Taylor, Memphis, TN (US)

(21) Appl. No.: 13/460,114

(22) Filed: Jun. 18, 2012

Related U.S. Application Data
(60) Provisional application No. 61/521,901, filed on Aug. 10, 2011.

(51) Int. Cl. A61F 7/08; A61F 13/56; A61F 13/493

(52) U.S. Cl. 604/385.16

(57) ABSTRACT

Panty pads are new innovative disposable underwear worn by women and girls during their menstrual cycles. It contains therapeutic heat to help relieve symptoms of PMS. Panty Pads eliminate the need for women and girls to soil or ruin expensive underwear worn during their menstrual cycles. This all in one disposable undergarment provides comfort and reliability.

### CROSS REFERENCE TO RELATED APPLICATION

This Panty Pads application claim priority, Ser. No. 61/521,901, filed on Aug. 10, 2011.

### STATEMENT REGARDING FEDERALLY SPONSORED RESEARCH OR DEVELOPMENT

Not Applicable

### REFERENCE TO SEQUENCE LISTING A TABLE OR A COMPUTER PROGRAM LISTING COMPACT DISC APPENDIX

Not Applicable

### BACKGROUND OF INVENTION

Panty Pads are disposable underwear designed for women and girls to wear during their menstrual cycles. Traditional pads in the marketplace tend to be bulky or do not stay in place. This often causes frustration with women and girls alike because expensive underwear is soiled or ruined. Panty Pads is a more effective approach and solution to this problem as well as to problems of premenstrual syndrome PMS. Therapeutic heat which is optional throughout areas of the panty pads soothes and eases cramps and bloating. Medical practitioners as well as manufactures would benefit from a product like the Panty Pads. Comfort and reliability are provided to women and girls during their menstrual cycles with the Panty Pads. People who have to endure long stays in hospitals can also benefit from this invention.

### BRIEF SUMMARY OF THE INVENTION

Panty Pads are a new innovative approach to women and girls menstrual cycles. Panty Pads solve the problem of women ruining and soiling their expensive underwear during their menstrual cycles. This undergarment is designed with optional therapeutic heat. This therapeutic heat can be placed throughout the panty with strips patches or bands. Panty Pads are created to fit just as comfortable as a pair of traditional panties. This revolutionary underwear solves many problems women and girls face when dealing with their menstrual cycles. Traditional pads tend to be bulky or do not stay in place. Panty Pads new design eliminates the need for pads and panties to be worn separately. This all in one disposable panty provides comfort and reliability to women and girls during their cycles. Women of all ages will appreciate the convenience of this inventions sanitation and practicality.

### BRIEF DESCRIPTION OF THE SEVERAL VIEWS OF THE DRAWINGS

FIG. 1 shows the invention laid flat.

FIG. 2 shows the padded crotch section of Panty Pads.

FIG. 3 shows the completed product with side fasteners.

FIG. 4 shows the therapeutic band at the front.

FIG. 5 shows the variety of potential colors and styles.

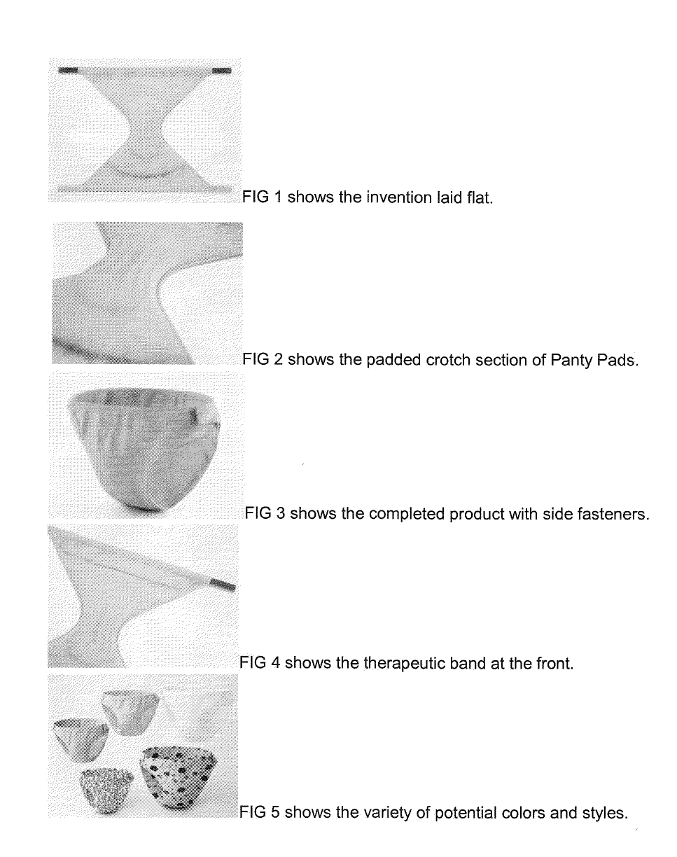

The forgoing descriptions of specific embodiments of the present invention have been presented for purpose of illustration and description. They are not intended to be exhaustive or to limit the present invention to the precise forms disclosed and obviously many modifications and variations are possible in light of the above teaching. The exemplary embodiment was chosen and described in order to best explain the principles of the present invention and its practical application to thereby enable others skilled in the art to best utilize the present invention and various embodiments with various modifications as are suited to the particular use contemplated.

### DETAILED DESCRIPTION TO THE INVENTION

Panty pads are disposable undergarments that can replace traditional underwear worn by women and girls during their menstrual cycles. The Panty Pads provide comfort and discreetness to women during their menstrual cycles. They are designed to look and feel just as comfortable as any other pair of panties a woman could wear. The crotch area of this disposable garment could be lined with absorbent material. A therapeutic band patch or pad could be placed along and under the elastic waistband to the top of the crotch area. Placement of the therapeutic heat is optional throughout the Panty Pads. The rest of the product could be held together by thin mesh or other suitable material. Panty pads are connected at the hips left and right sides by Velcro plastic tabs or hook fasteners. These panties come in varying sizes and absorbencies.

What is claimed

1. Panty pads is a disposable undergarment with inner and outer layers of expandable moveable material with contiguous therapeutic heat throughout the panty that is optional in placement for women and girls to wear during their menstrual cycles or as determined comprising substantially the body consisting of a front panel or portion a back or rear panel or portion an absorbent padded crotch area a stretchable left side front and back leg opening a right side front and back leg opening a waistband that borders the upper front and back or rear panel or portion a contiguous therapeutic heat patch pad band gel or strip throughout the entire panty which provide optional placement of the therapeutic heat to relieve cramps or symptoms of pms or other aliments a front and back panel or portion left side and right side fasteners that attach and hold together the panty to give support and easy access to apply or dispose the panty.

2. the disposable undergarment of claim 1 for women and girls to wear wherein a front panel or portion comprises a breathable expandable or moveable inner and outer layer of material with optional placement of contiguous therapeutic heat patches pads bands gels or strips while allowing attachment of a stretchable waistband and left and right side leg openings

3. the disposable undergarment of claim 1 for women and girls to wear wherein a back or rear panel or portion comprises a breathable expandable or moveable inner and outer layer of material with optional placement of contiguous therapeutic heat patches pads bands gels or strips while allowing attachment of a stretchable waistband and left and right side leg openings

4. the disposable undergarment of claim 1 for women and girls to wear consist of a disposed predetermined absorbent padded crotch area sandwiching the bodies front and back panels or portions along with front and back left and right side stretchable leg openings

5. the disposable undergarment of claim 1 for women and girls to wear wherein heretofore a front and back left side and right side leg opening is comprised of stretchable material contiguous to the front and back or rear panels or portions and an absorbent crotch area.

6. the disposable undergarment of claim 1 for women and girls to wear wherein a predetermined upper surrounding waistband of stretchable of moveable material is mended with front and back or rear panel or portion

7. the disposable undergarment of claim 1 for women and girls to wear offer a therapeutic heat patch pad band gel strip or related method of pain relief that is substantially contiguous to the entire panty throughout and is optional in its placement as related to the body as the front and back or rear panels or portions the crotch area the left and right side leg openings front and back and the waistband front and back or rear and fasteners

8. the disposable undergarment of claim 1 for women and girls to wear wherein various left and right side fasteners have a means for attaching front and back panels or portions.
